(19) United States
(12) Patent Application Publication
(10) Pub. No.: US 2024/0245590 A1
(43) Pub. Date: Jul. 25, 2024

(54) RESHAPING COMPOSITION FOR KERATIN FIBERS

(71) Applicant: KAO GERMANY GMBH, Darmstadt (DE)

(72) Inventors: Bernd NÖCKER, Darmstadt (DE); Steven BREAKSPEAR, Darmstadt (DE); Stephanie ILHAM, Darmstadt (DE)

(73) Assignee: KAO GERMANY GMBH, Darmstadt (DE)

(21) Appl. No.: 18/289,669

(22) PCT Filed: May 13, 2022
(86) PCT No.: PCT/EP2022/063069

§ 371 Date: Nov. 6, 2023

(30) Foreign Application Priority Data
May 18, 2021 (EP) 21174368.7

(51) Int. Cl. A61K 8/362; A61K 8/365; A61K 8/44; A61Q 5/04

(52) U.S. Cl. CPC A61K 8/362; A61K 8/365; A61K 8/44; A61K 8/447; A61Q 5/04; A61K 2800/88

(57) ABSTRACT

Aqueous reshaping compositions for keratin fibers, two-chamber packaging devices, kits-of-parts, and reshaping processes are provided, wherein said compositions have a pH in the range of 1 to 4 and comprise a) one or more bidentate or tridentate carboxylic acid, and/or their salt(s), and/or their hydrates, and/or their mixtures, and b) one or more one or more amino acid(s), and/or their salt(s), and/or mixtures of 1) and 2), wherein the total concentration of compound(s) according to group a) is 1% by weight or more, calculated to the total weight of the composition.

### FIELD OF THE INVENTION

The invention is directed to the provision of a reshaping composition for keratin fibers, a reshaping process, and a kit-of-parts for reshaping.

### BACKGROUND OF THE INVENTION

Straightening treatments for keratin fibers based on formaldehyde, strong alkali compositions (lanthionization) or reducing/oxidizing processes are highly effective, but these processes usually cause significant hair damage. In the past decade, treatments based on glyoxylic acid became more popular, but for some clients the straightening effect is too permanent or too strong.

WO2015/075062 discloses hair-straightening compositions comprising citric acid.

WO2013/174690 discloses hair-straightening compositions comprising citric acid and gluconolactone. The hair treated with heat.

WO2017/137182 discloses hair-straightening compositions comprising citric acid and silk sericin.

WO2013/174703 discloses hair-straightening compositions comprising citric acid and sugar alcohols. The hair treated with heat.

### SUMMARY OF THE INVENTION

The first object of the present invention is an aqueous reshaping composition for keratin fibers, preferably for human keratin fibers, more preferably for human hair, having a pH in the range of 1 to 6 and comprising:
a) one or more bidentate or tridentate carboxylic acid, and/or their salt(s), and/or their hydrates, and/or their mixtures,
b) one or more amino acid(s), and/or their salt(s), and/or mixtures of 1) and 2),
   wherein the total concentration of compound(s) according to group a) is 1% by weight or more, calculated to the total weight of the composition.

The second object of the present invention is a two-chamber packaging device having:
a first chamber comprising a first aqueous composition comprising one or more compound(s) according to group a) as defined above, and
a second chamber comprising a second aqueous composition comprising one or more compound(s) according to group b) as defined above.

The third object of the present invention is a kit-of-parts having a first component with an aqueous composition comprising one or more compound(s) according to group a) as defined above, and
a second component with an aqueous composition comprising one or more compound(s) according to group b) as defined above.

The fourth object of the present invention is a reshaping process for keratin fibers comprising the steps in the following order of:
i) providing the composition as defined above for the reshaping composition, or providing and mixing the first and second component as defined above for the kit, or providing and dispensing and optionally mixing the compositions as defined above for the two-chamber packaging, and yielding a composition having a pH in the range of 1 to 6, preferably in the range of 1.25 to 5, more preferably in the range of 1.5 to 4.5, further more preferably in the range of 2 to 4,
ii) applying the composition of step i) onto keratin fibers,
iii) optionally covering the keratin fibers with a moisture barrier,
iv) leaving the composition onto the keratin fibers for a time period of 1 min to 24 h, preferably for a time period of 1 min to 120 min,
v) optionally rinsing-off the hair,
vi) drying the hair,
vii) applying heat to the keratin fibers at a temperature in the range of 50° ° C. to 230° C.,
viii) rinsing-off the keratin fibers and optionally drying the keratin fibers.

### DETAILED DESCRIPTION OF THE INVENTION

Inventors of the present invention have unexpectedly found out that a composition according to claim 1 effectively straightens hair. The straightening effect lasted over at least 10 hair washes. Furthermore, the hair was less damaged in comparison to known other straightening techniques.

### Aqueous Reshaping Composition

The present invention is directed to an aqueous reshaping composition for keratin fibers, preferably for human keratin fibers, more preferably for human hair, having a pH in the range of 1 to 6 and comprising:
a) one or more bidentate or tridentate carboxylic acid, and/or their salt(s), and/or their hydrates, and/or their mixtures,
b) one or more amino acid(s), and/or their salt(s),
   wherein the total concentration of compound(s) according to group a) is 1% by weight or more, calculated to the total weight of the composition.

The term ‘aqueous’ within the meaning of the present invention requires the composition to have a water content of 50% by weight or more, preferably to have a water content of 60% by weight or more, more preferably to have a water content of 70% by weight or more, calculated to the total weight of the composition.

It is preferred from the viewpoint of straightening efficiency that the pH of the composition is 5 or lower, preferably the pH is 4.5 or lower, more preferably the pH is 4 or lower.

It is preferred from the viewpoint of cosmetic safety that the pH of the composition is 1.25 or higher, preferably 1.5 or higher, further more preferably 2 or higher.

For attaining the above-mentioned effects, it is preferred that the pH of the composition is in the range of 1.25 to 5, more preferably in the range of 1.5 to 4.5, further more preferably in the range of 2 to 4.

It is preferred from the viewpoint of performance that the composition of the present invention is a hair straightening composition.

It is preferred from the viewpoint of minimizing damage to keratin fibers that the composition comprises less than 1% by weight of oxidizing agent(s), preferably less than 1% by weight of oxidizing agent(s), calculated to the total weight of the composition.

It is preferred from the viewpoint of minimizing damage to keratin fibers that the composition is free of oxidizing agents.

It is further preferred from the viewpoint of cosmetic applicability that the composition of the present invention is a temporary reshaping composition for keratin fibers, preferably a temporary reshaping composition for keratin fibers with heat at 50° C. or above, more preferably with heat at 120° C. or above.

It is further preferred from the viewpoint of cosmetic applicability that the composition of the present invention is a temporary straightening composition.

### Compound(s) According to Group a)

The composition of the present invention comprises one or more bidentate or tridentate carboxylic acid, and/or their salt(s), and/or their mixtures, as compound(s) according to group a).

Suitable compound(s) according to group a) is/are bidentate carboxylic acid(s), preferably it is oxalic acid, malonic acid, succinic acid, glutaric acid, adipic acid, pimelic acid, suberic acid, azelaic acid, sebacic acid, maleic acid, fumaric acid, acetylenedicarboxylic acid, glutaconic acid, traumatic acid, tartronic acid, mesoxalic acid, malic acid, tartraric acid, oxaliacetic acid, dioxosuccinic acid, glutaric acid, ketoglutaric acid, and/or their salt(s), and/or their hydrates, and/or their mixtures.

The more preferred compound(s) according to group a) from the viewpoint of cosmetic acceptance is/are selected from oxalic acid, malonic acid, succinic acid, maleic acid, fumaric acid, and/or their salt(s), and/or their hydrates, and/or their mixtures.

The most preferred compound(s) according to group a) from the viewpoint of cosmetic acceptance is/are according to group a) is/are succinic acid, and/or its salt(s), and/or its hydrate(s).

Further suitable compound(s) according to group a) is/are tridentate carboxylic acid(s), preferably it is/are citric acid, isocitric acid, aconitic acid, propane-1,2,3-tricarboxylic acid, trimesic acid, and/or their salt(s), and/or their hydrates, and/or their mixtures.

More preferably one or more compound(s) according to group a) is/are selected from citric acid, isocitric acid, aconitic acid, propane-1,2,3-tricarboxylic acid, trimesic acid, and/or their salt(s), and/or their hydrates, and/or their mixtures, form the viewpoint of straightening efficiency.

The more preferred compound(s) according to group a) from the viewpoint of straightening efficiency is/are selected from citric acid or isocitric acid, and/or their salt(s), and/or their hydrates, and/or their mixtures.

The most preferred compound(s) according to group a) is citric acid, and/or its salt(s), and/or its hydrates.

It is preferred from the viewpoint of straightening efficiency that the total concentration of compound(s) according to group a) is 1% by weight or more, more preferably it is 2% by weight or more, further more preferably it is 3% by weight or more, calculated to the total weight of the composition.

It is further preferred from the viewpoint of cosmetic safety that the total concentration of compound(s) according to group a) is 30% by weight or less, preferably 20% by weight or less, more preferably 15% by weight or less, still further more preferably 10% by weight or less, still further more preferably 8% by weight or less.

For attaining the above-mentioned effects, it is preferred that the total concentration of compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

In one aspect of the present invention, the total concentration of bidentate carboxylic acid(s) and/or their salt(s), and/or their hydrates, and/or their mixtures, as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

In one aspect of the present invention, the total concentration of succinic acid and/or its salt(s) and/or its hydrates as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

In one aspect of the present invention, the total concentration of tridentate carboxylic acid(s) and/or their salt(s), and/or their hydrates, and/or their mixtures, as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

In one aspect of the present invention, the total concentration of citric acid and/or its salt(s) and/or its hydrates as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

### Compound(s) According to Group b)

The composition of the present invention comprises one or more amino acid(s), and/or their salt(s), and/or mixtures of 1) and 2), as compound(s) according to group b).

Suitable compound(s) according to group b) is/are serine, threonine, cysteine, cystine, alanine, glycine, proline, and/or valine, and/or their mixtures, and/or their salt(s). It is preferred that one or more compound(s) according to group b) is serine, threonine, cysteine, cystine, and/or their mixtures, and/or their salt(s), still more preferably it is serine and/or threonine, and/or their mixtures, and/or their salt(s).

The most preferred compound(s) according to group b) is serine and/or its salt(s).

Another preferred compound(s) according to group b) is cysteine and/or its salt(s).

It is further preferred from the viewpoint of straightening efficiency that the weight ratio of compound(s) according to a) to compound(s) according to b) is in the range of 0.2 to 5, preferably it is in the range of 0.25 to 3, more preferably it is in the range of 0.5 to 2, further more preferably it is in the range of 0.5 to 1.

It is preferred from the viewpoint of straightening efficiency that the total concentration of one or more compound(s) according to group b) is 0.5% by weight or more, preferably it is 1% by weight or more, further more preferably it is 2% by weight or more, calculated to the total weight of the composition.

It is preferred from the viewpoint of cosmetic safety that the total concentration of one or more compound(s) according to group b) is 15% by weight or less, preferably it is 10% by weight or less, further more preferably it is 8% by weight or less, calculated to the total weight of the composition.

For attaining the above-mentioned effects, it is preferred that the total concentration of one or more compound(s) according to group b) is in the range of 0.5% to 15% by weight, preferably in the range of 1% to 10% by weight, further more preferably in the range of 2% to 8% by weight, calculated to the total weight of the composition.

The disclosure of the present invention is also directed to an aqueous reshaping composition for keratin fibers, preferably for human keratin fibers, more preferably for human hair, having a pH in the range of 1 to 6 and comprising:
a) one or more tridentate carboxylic acid, and/or their salt(s), and/or their hydrates, and/or their mixtures, preferably it is citric acid and/or its salt(s), and/or its hydrates,
b) one or more compound(s) selected from:
1) one or more trialcohol(s), preferably selected from glycerol,
2) one or more amino acid(s), and/or their salt(s), preferably selected from serin and/or its salt(s), and/or their mixtures,
   wherein the total concentration of compound(s) according to group a) is 1% by weight or more, preferably it is in the range of 1% to 30% by weight, more preferably in the range of 2% to 20% by weight, further more preferably in the range of 3% to 15% by weight, still further more preferably in the range of 3% to 10% by weight, still further more preferably in the range of 3% to 8% by weight, calculated to the total weight of the composition.

### Compound(s) According to Group c)

It is preferred from the viewpoint of conditioning that it comprises one or more conditioning compound(s) as compound(s) according to group c), preferably selected from silicones, fatty alcohols, and/or cationic surfactants, and/or their salt(s), and/or their mixtures.

Suitably, one or more compound(s) according to group c) is/are silicones, preferably aminated silicones.

It is also preferred that one or more compound(s) according to group c) is/are cationic surfactant(s), and/or their salt(s), preferably selected from quaternary ammonium surfactants and/or amidopropylamino surfactants, and/or their salt(s), and/or their mixtures.

Suitable compounds are cetrimonium chloride, behentrinonium chloride, behenamidopropyl dimethylamine, and/or their salt(s), and/or their mixtures.

It is preferred from the viewpoint of conditioning effect that the total concentration of one or more compound(s) according to group c) is in the range of 0.1% to 5% by weight, preferably in the range of 0.25% to 3% by weight, more preferably in the range of 0.5% to 2% by weight, calculated to the total weight of the composition.

In one aspect of the present invention that the total concentration of silicones, preferably aminated silicones, as one or more compound(s) according to group c) is in the range of 0.1% to 5% by weight, preferably in the range of 0.25% to 3% by weight, more preferably in the range of 0.5% to 2% by weight, calculated to the total weight of the composition.

In one aspect of the present invention that the total concentration of cationic surfactant(s), and/or their salt(s), preferably selected from quaternary ammonium surfactants and/or amidopropylamino surfactants, and/or their salt(s), and/or their mixtures, as one or more compound(s) according to group c) is in the range of 0.1% to 5% by weight, preferably in the range of 0.25% to 3% by weight, more preferably in the range of 0.5% to 2% by weight, calculated to the total weight of the composition.

### Two-Chamber Packaging

The present invention is also directed to a two-chamber packaging device having:
a first chamber comprising a first aqueous composition comprising one or more compound(s) according to group a) as defined above, and
a second chamber comprising a second aqueous composition comprising one or more compound(s) according to group b) as defined above.

The first and second chamber compositions are kept separate until directly prior to use onto keratin fibers. The mixture of the compositions has a pH in the range of 1 to 6, preferably in the range of 1.25 to 5, more preferably in the range of 1.5 to 4.5, further more preferably in the range of 2 to 4.

From this viewpoint, it is preferred that the first and second chamber compositions have a pH in the range of 1 to 6, preferably in the range of 1.25 to 5, more preferably in the range of 1.5 to 4.5, further more preferably in the range of 2 to 4.

In another aspect, the first chamber composition has a pH in the range of 1 to 6, preferably in the range of 1.25 to 5, more preferably in the range of 1.5 to 4.5, further more preferably in the range of 2 to 4, and the second chamber composition has a pH in the range of 7 to 12. However, the mixtures of the compositions obtained directly prior to application onto keratin fibers has a pH in the range of 1 to 6, preferably in the range of 1.25 to 5, more preferably in the range of 1.5 to 4.5, further more preferably in the range of 2 to 4.

In another aspect, the first chamber composition has a pH in the range of 7 to 12, and the second chamber composition has a pH in the range of 1 to 6, preferably in the range of 1.25 to 5, more preferably in the range of 1.5 to 4.5, further more preferably in the range of 2 to 4. However, the mixtures of the compositions obtained directly prior to application onto keratin fibers has a pH in the range of 1 to 6, preferably in the range of 1.25 to 5, more preferably in the range of 1.5 to 4.5, further more preferably in the range of 2 to 4.

In one aspect of the present invention, the packaging container comprises a valve configured to subsequently or simultaneously dispense from two chambers upon actuation.

The packaging container may be made of any suitable material, for example any suitable metal, single constituent or composite plastics.

### Kit-of-Parts

The present invention is also directed to a kit-of-parts having a first component with an aqueous composition comprising one or more compound(s) according to group a) as defined above, and
a second component with an aqueous composition comprising one or more compound(s) according to group b) as defined above.

The first and second components are kept separate until directly prior to use onto keratin fibers. The mixture of the components has a pH in the range of 1 to 6, preferably in the range of 1.25 to 5, more preferably in the range of 1.5 to 4.5, further more preferably in the range of 2 to 4.

From this viewpoint, it is preferred that the first and second components of the kit have a pH in the range of 1 to 6, preferably in the range of 1.25 to 5, more preferably in the range of 1.5 to 4.5, further more preferably in the range of 2 to 4.

In another aspect, the first component of the kit has a pH in the range of 1 to 6, preferably in the range of 1.25 to 5, more preferably in the range of 1.5 to 4.5, further more preferably in the range of 2 to 4, and the second component of the kit has a pH in the range of 7 to 12. However, the mixtures of the components obtained directly prior to application onto keratin fibers has a pH in the range of 1 to 6, preferably in the range of 1.25 to 5, more preferably in the range of 1.5 to 4.5, further more preferably in the range of 2 to 4.

In another aspect, the first component of the kit has a pH in the range of 7 to 12, and the second component has a pH in the range of 1 to 6, preferably in the range of 1.25 to 5, more preferably in the range of 1.5 to 4.5, further more preferably in the range of 2 to 4. However, the mixtures of the components obtained directly prior to application onto keratin fibers has a pH in the range of 1 to 6, preferably in the range of 1.25 to 5, more preferably in the range of 1.5 to 4.5, further more preferably in the range of 2 to 4.

It is preferred from the viewpoint of conditioning that the first component and/or the second component further comprise one or more compound(s) according to group c) as defined above. The concentration ranges for the compound(s) according to group c) as defined above are applicable to the individual components of the kit.

It is preferred from the viewpoint of minimizing damage to keratin fibers that the first component comprise less than 1% by weight of reducing agent(s), preferably less than 0.1% by weight of reducing agent(s), calculated to the total weight of the composition, more preferably they are free of reducing agents.

It is further preferred from the viewpoint of minimizing damage to keratin fibers that the first and second component comprise less than 1% by weight of oxidizing agent(s), preferably less than 1% by weight of oxidizing agent(s), calculated to the total weight of the composition, more preferably they are free of oxidizing agents.

### Reshaping Process

The present invention is also directed to a reshaping process for keratin fibers comprising the steps in the following order of:
i) providing the composition as defined above for the reshaping composition, or providing and mixing the first and second component as defined above for the kit, or providing and dispensing and optionally mixing the compositions as defined above for the two-chamber packaging, and yielding a composition having a pH in the range of 1 to 6, preferably in the range of 1.25 to 5, more preferably in the range of 1.5 to 4.5, further more preferably in the range of 2 to 4,
ii) applying the composition of step i) onto keratin fibers,
iii) optionally covering the keratin fibers with a moisture barrier,
iv) leaving the composition onto the keratin fibers for a time period of 1 min to 120 min,
v) optionally rinsing-off the hair,
vi) drying the hair,
vii) applying heat to the keratin fibers at a temperature in the range of 50° C. to 230° C.,
viii) rinsing-off the keratin fibers and optionally drying the keratin fibers.

It is preferred from the viewpoint of cosmetic applicability that the moisture barrier of step iii) is aluminium foil or a plastic wrapping.

It is further preferred that the compositions in step ii) are applied onto dry keratin fibers.

It is preferred from the viewpoint of reshaping performance that the temperature of step vii) is in the range of 90° C. to 230° C., preferably in the range of 120° C. to 230° C., more preferably in the range of 150° ° C. to 230° C.

It is preferred from the viewpoint of cosmetic applicability that it is a process for temporary reshaping keratin fibers, preferably for temporary straightening keratin fibers.

It is preferred from the viewpoint of reshaping performance that the time period of step iv) is in the range of 2 min to 60 min, preferably in the range of 5 min to 45 min, more preferably in the range of 10 min to 40 min.

It is preferred from the viewpoint of cosmetic applicability that heat in step vii) is applied with a straightening iron.

The disclosure of the present invention is also directed to:

<1> An aqueous reshaping composition for keratin fibers, preferably for human keratin fibers, more preferably for human hair, having a pH in the range of 1 to 6 and comprising:
a) one or more bidentate or tridentate carboxylic acid, and/or their salt(s), and/or their mixtures,
b) one or more compound(s) amino acid(s), and/or their salt(s), and/or mixtures,
   wherein the total concentration of compound(s) according to group a) is 1% by weight or more, calculated to the total weight of the composition.

<2> The composition of clause <1>, characterized in that one or more compound(s) according to group a) is/are a bidentate carboxylic acid(s), preferably it is oxalic acid, malonic acid, succinic acid, glutaric acid, adipic acid, pimelic acid, suberic acid, azelaic acid, sebacic acid, maleic acid, fumaric acid, acetylenedicarboxylic acid, glutaconic acid, traumatic acid, tartronic acid, mesoxalic acid, malic acid, tartraric acid, oxaliacetic acid, dioxosuccinic acid, glutaric acid, ketoglutaric acid, and/or their salt(s), and/or their hydrates, and/or their mixtures.

<3> The composition according to any of the clauses <1> and/or <2> characterized in that one or more compound(s) according to group a) is/are selected from oxalic acid, malonic acid, succinic acid, maleic acid, fumaric acid, and/or their salt(s), and/or their hydrates, and/or their mixtures.

<4> The composition according to any of the clauses <1> to <3> characterized in that one or more compound(s) according to group a) is/are succinic acid, and/or its salt(s), and/or its hydrate(s).

<5> The composition according to any of the clauses <1> to <4> characterized in that one or more compound(s) according to group a) is/are a tridentate carboxylic acid, and/or its/their salt(s), and/or its/their hydrates.

<6> The composition according to any of the clauses <1> and/or <5> characterized in that one or more tridentate carboxylic acid(s) as compound(s) according to group a) is/are citric acid, isocitric acid, aconitic acid, propane-1,2,3-tricarboxylic acid, trimesic acid, and/or their salt(s), and/or their hydrates, and/or their mixtures.

<7> The composition according to any of the clauses <1> to <6> characterized in that one or more compound(s) according to group a) is/are citric acid, isocitric acid, aconitic acid, propane-1,2,3-tricarboxylic acid, trimesic acid, and/or their salt(s), and/or their hydrates, and/or their mixtures.

<8> The composition according to any of the clauses <1> to <7> characterized in that one or more compound(s) according to group a) is/are selected from citric acid or isocitric acid, and/or their salt(s), and/or their hydrates, and/or their mixtures.

<9> The composition according to any of the clauses <1> to <8> characterized in that one or more compound(s) according to group a) is citric acid, and/or its salt(s), and/or its hydrates.

<10> The composition according to clause <1> characterized in that one or more compound(s) according to group a) is/are a bidentate carboxylic acid(s) and/or its/their salt(s), and/or its/their hydrates, preferably it is oxalic acid, malonic acid, succinic acid, glutaric acid, adipic acid, pimelic acid, suberic acid, azelaic acid, sebacic acid, maleic acid, fumaric acid, acetylenedicarboxylic acid, glutaconic acid, traumatic acid, tartronic acid, mesoxalic acid, malic acid, tartraric acid, oxaliacetic acid, dioxosuccinic acid, glutaric acid, ketoglutaric acid, and/or their salt(s), and/or their hydrates, and/or their mixtures, or one or more tridentate carboxylic acid(s), preferably citric acid, isocitric acid, aconitic acid, propane-1,2,3-tricarboxylic acid, trimesic acid, and/or their salt(s), and/or their hydrates, and/or their mixtures.

<11> The composition according to any of the clauses <1> to <10> characterized in that one or more compound(s) according to group a) is/are is/are succinic acid, and/or its salt(s), and/or its hydrate(s), citric acid, and/or its salt(s), and/or its hydrates, and/or their mixtures.

<12> The composition according to any of the clauses <1> to <11> characterized in that the total concentration of compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

<13> The composition according to any of the clauses <1> to <12> characterized in that the total concentration of bidentate carboxylic acid(s) and/or their salt(s), and/or their hydrates, and/or their mixtures, as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

<14> The composition according to any of the clauses <1> to <13> characterized in that the total concentration of succinic acid and/or its salt(s) and/or its hydrates as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

<15> The composition according to any of the clauses <1> to <14> characterized in that the total concentration of tridentate carboxylic acid(s) and/or their salt(s), and/or their hydrates, and/or their mixtures, as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

<16> The composition according to any of the clauses <1> to <15> characterized in that the total concentration of citric acid and/or its salt(s), and/or its hydrates, as compound(s) according to group a) is in the range of 1% to 30% by weight, preferably in the range of 2% to 20% by weight, more preferably in the range of 3% to 15% by weight, further more preferably in the range of 3% to 10% by weight, still further more preferably in the range of 3% to 8% by weight, calculated to the total weight of the composition.

<17>. The composition according to any of the clauses <1> to <16> characterized in that one or more compound(s) according to group b) is/are serine, threonine, cysteine, cystine, alanine, glycine, proline, and/or valine, and/or their mixtures, and/or their salt(s).

<18> The composition according to any of the clauses <1> to <17> characterized in that one or more compound(s) according to group b) is serine, threonine, cysteine, cystine, and/or their mixtures, and/or their salt(s) more preferably it is serine and/or threonine, and/or their mixtures, and/or their salt(s).

<19> The composition according to any of the clauses <1> to <18> characterized in that one or more compound(s) according to group b) is serine and/or its salt(s).

<20> The composition according to any of the clauses <1> to <19> characterized in that one or more compound(s) according to group b) is cysteine and/or its salt(s).

<21> The composition according to any of the clauses <1> to <20> characterized in that one or more compound(s) according to group a) is citric acid and/or its salt(s), and/or its hydrates, and one or more compound(s) according to group b) is serin and/or its salt(s).

<22> The composition according to any of the clauses <1> to <20> characterized in that one or more compound(s) according to group a) is citric acid and/or its salt(s), and/or its hydrates, and one or more compound(s) according to group b) is cysteine and/or its salt(s).

<23> The composition according to any of the clauses <1> to <22> characterized in that the weight ratio of compound(s) according to a) to compound(s) according to b) is in the range of 0.2 to 5, preferably it is in the range of 0.25 to 3, more preferably it is in the range of 0.5 to 2, further more preferably it is in the range of 0.5 to 1.

<24> The composition according to any of the clauses <1> to <23> characterized in that the total concentration of one or more compound(s) according to group b) is in the range of 0.5% to 15% by weight, preferably in the range of 1% to 10% by weight, further more preferably in the range of 2% to 8% by weight, calculated to the total weight of the composition.

<25> The composition according to any of the clauses <1> to <23> characterized in that it comprises one or more conditioning compound(s) as compound(s) according to group c), preferably selected from silicones, fatty alcohols, and/or cationic surfactants, and/or their salt(s), and/or their mixtures.

<26> The composition according to clause <25> characterized in that one or more compound(s) according to group c) is/are silicones, preferably aminated silicones.

<27> The composition according to any of the clauses <25> to <26> characterized in that one or more compound(s) according to group c) is/are cationic surfactant(s), and/or their salt(s), preferably selected from quaternary ammonium surfactants and/or amidopropylamino surfactants, and/or their salt(s), and/or their mixtures.

<28> The composition according to any of the clauses <25> to <27> characterized in that the total concentration of one or more compound(s) according to group c) is in the range of 0.1% to 5% by weight, preferably in the range of 0.25% to 3% by weight, more preferably in the range of 0.5% to 2% by weight, calculated to the total weight of the composition.

<29> The composition according to any of the clauses <25> to <27> characterized in that the total concentration of silicones, preferably aminated silicones, as one or more compound(s) according to group c) is in the range of 0.1% to 5% by weight, preferably in the range of 0.25% to 3% by weight, more preferably in the range of 0.5% to 2% by weight, calculated to the total weight of the composition.

<30> The composition according to any of the clauses <25> to <27> characterized in that the total concentration of cationic surfactant(s), and/or their salt(s), preferably selected from quaternary ammonium surfactants and/or amidopropylamino surfactants, and/or their salt(s), and/or their mixtures, as one or more compound(s) according to group c) is in the range of 0.1% to 5% by weight, preferably in the range of 0.25% to 3% by weight, more preferably in the range of 0.5% to 2% by weight, calculated to the total weight of the composition.

<31> The composition according to any of the clauses <1> to <30> characterized in that the composition is a hair straightening composition.

<32> The composition according to any of the clauses <1> to <31> characterized in that the composition is free of reducing agents.

<33> The composition according to any of the clauses <1> to <32> characterized in that the composition comprises less than 1% by weight of oxidizing agent(s), preferably less than 1% by weight of oxidizing agent(s), calculated to the total weight of the composition.

<34> The composition according to any of the clauses <1> to <33> characterized in that the composition is free of oxidizing agents.

<35> The composition according to any of the clauses <1> to <34> characterized in that it is a temporary reshaping composition for keratin fibers, preferably a temporary reshaping composition for keratin fibers with heat at 50° C. or above, more preferably with heat at 120° C. or above.

<36> The composition according to any of the clauses <1> to <35> characterized in that it is a temporary straightening composition.

<37> The composition according to any of the clauses <1> to <36> characterized in that it comprises water at 50% by weight or more, preferably to have a water content of 60% by weight or more, more preferably to have a water content of 70% by weight or more, calculated to the total weight of the composition.

<38> The composition according to any of the clauses <1> to <37> characterized in that the pH of the composition is in the range of 1.25 to 5, more preferably in the range of 1.5 to 4.5, further more preferably in the range of 2 to 4.

The disclosure of the present invention is also directed to:

<39> A two-chamber packaging device having:
a first chamber comprising a first aqueous composition comprising one or more compound(s) according to group a) as defined in any of the clauses <1> to <38>, and
a second chamber comprising a second aqueous composition comprising one or more compound(s) according to group b) as defined in any of the clauses <1> to <38>.

<40> The packaging according to clause <39> characterized in that the first and second chamber compositions are kept separate until directly prior to use onto keratin fibers.

<41> The packaging according to any of the clauses <39> to <40> characterized in that the mixture of the compositions has a pH in the range of 1 to 6, preferably in the range of 1.25 to 5, more preferably in the range of 1.5 to 4.5, further more preferably in the range of 2 to 4.

<42> The packaging according to any of the clauses <39> to <41> characterized in that the first and second chamber compositions have a pH in the range of 1 to 6, preferably in the range of 1.25 to 5, more preferably in the range of 1.5 to 4.5, further more preferably in the range of 2 to 4.

<43> The packaging according to any of the clauses <39> to <41> characterized in that the first chamber composition has a pH in the range of 1 to 6, preferably in the range of 1.25 to 5, more preferably in the range of 1.5 to 4.5, further more preferably in the range of 2 to 4, and the second chamber composition has a pH in the range of 7 to 12.

<44> The packaging according to any of the clauses <39> to <41> characterized in that the first chamber composition has a pH in the range of 7 to 12, and the second chamber composition has a pH in the range of 1 to 6, preferably in the range of 1.25 to 5, more preferably in the range of 1.5 to 4.5, further more preferably in the range of 2 to 4.

<45> The packaging according to any of the clauses <39> to <44> characterized in that the packaging comprises a valve configured to subsequently or simultaneously dispense from two chambers upon actuation.

<46> The packaging according to clause <45> characterized in that the packaging comprises a valve configured to simultaneously dispense from two chambers upon actuation.

The disclosure of the present invention is also directed to:

<47> A kit-of-parts having a first component with an aqueous composition comprising one or more compound(s) according to group a) as defined in any of the clauses <1> to <38>, and
a second component with an aqueous composition comprising one or more compound(s) according to group b) as defined in any of the clauses <1> to <38>.

<48> The kit-of-parts according to clause <47> characterized in that the first and second components are kept separate until directly prior to use onto keratin fibers.

<49> The kit-of-parts according to any of the clauses <47> to <48> characterized in that the mixture of the components has a pH in the range of 1 to 6, preferably in the range of 1.25 to 5, more preferably in the range of 1.5 to 4.5, further more preferably in the range of 2 to 4.

<50> The kit-of-parts according to any of the clauses <47> to <49> characterized in that the first and second components of the kit have a pH in the range of 1 to 6, preferably in the range of 1.25 to 5, more preferably in the range of 1.5 to 4.5, further more preferably in the range of 2 to 4.

<51> The kit-of-parts according to any of the clauses <47> to <49> characterized in that the first component of the kit has a pH in the range of 1 to 6, preferably in the range of 1.25 to 5, more preferably in the range of 1.5 to 4.5, further more preferably in the range of 2 to 4, and the second component of the kit has a pH in the range of 7 to 12.

<52> The kit-of-parts according to any of the clauses <47> to <49> characterized in that the first component of the kit has a pH in the range of 7 to 12, and the second component has a pH in the range of 1 to 6, preferably in the range of 1.25 to 5, more preferably in the range of 1.5 to 4.5, further more preferably in the range of 2 to 4.

<53> The kit-of-parts according to any of the clauses <47> to <52> characterized in that the first component and/or the second component further comprise one or more compound(s) according to group c) as defined in any of the clauses <25> to <30>.

<54> The kit-of-parts according to any of the clauses <47> to <53> characterized in that the first component comprises less than 1% by weight of reducing agent(s), preferably less than 0.1% by weight of reducing agent(s), calculated to the total weight of the composition, more preferably they are free of reducing agents.

<55> The kit-of-parts according to any of the clauses <47> to <54> characterized in that the first and second component comprise less than 1% by weight of oxidizing agent(s), preferably less than 1% by weight of oxidizing agent(s), calculated to the total weight of the composition, more preferably they are free of oxidizing agents.

The disclosure of the present invention is also directed to:

<56> A reshaping process for keratin fibers comprising the steps in the following order of:
i) providing the composition as defined in any of the clauses <1> to <38>, or providing and mixing the first and second component as defined in any of the clauses <47> to <55>, or providing and dispensing and optionally mixing the compositions as defined in any of the clauses <39> to <46>, and yielding a composition having a pH in the range of 1 to 6, preferably in the range of 1.25 to 5, more preferably in the range of 1.5 to 4.5, further more preferably in the range of 2 to 4,
ii) applying the composition of step i) onto keratin fibers,
iii) optionally covering the keratin fibers with a moisture barrier,
iv) leaving the composition onto the keratin fibers for a time period of 1 min to 24 h, preferably for a time period of 1 min to 120 min,
v) optionally rinsing-off the hair,
vi) drying the hair,
vii) applying heat to the keratin fibers at a temperature in the range of 50° C. to 230° C.,
viii) rinsing-off the keratin fibers and optionally drying the keratin fibers.

<57> The process according to clause <56> characterized in that the temperature of step vii) is in the range of 90° C. to 230° C., preferably in the range of 120° C. to 230° ° C., more preferably in the range of 150° ° C. to 230° C.

<58> The process according to any of the clauses <56> to <57> characterized in that it is a process for temporary reshaping keratin fibers, preferably for temporary straightening keratin fibers.

<59> The process according to any of the clauses <56> to <58> characterized in that the time period of step iv) is in the range of 2 min to 60 min, preferably in the range of 5 min to 45 min, more preferably in the range of 10 min to 40 min.

<60> The process according to any of the clauses <56> to <59> characterized in that heat in step vii) is applied with a straightening iron.

The following examples are to illustrate the invention, but not to limit it.

### Examples

TABLE 1
The following compositions were prepared by subsequently
dissolving the compounds in water.

| | Inv. | Inv. | Inv. | Inv. | Inv. | Comp. | Comp. |
|---|---|---|---|---|---|---|---|
| | ex. 1 | ex. 2 | ex. 3 | ex. 4 | ex. 5 | ex. 1 | ex. 2 |
| Ingredient | [% by weight] | | | | | | |
| Succinic acid | 2.5 | 5.0 | 5.0 | 5.0 | 5.0 | — | 5.0 |
| Serine | 5.0 | 5.0 | 2.5 | 10.0 | — | 5.0 | — |
| Cysteine | — | — | — | — | 2.5 | — | — |
| NaOH | pH ad 2.5 | | | | | | |
| Water | Ad 100.0 | | | | | | |
| Durability after | 0.22 | 0.50 | 0.27 | 0.23 | 0.81 | 0.01 | 0.06 |
| 10 washes | | | | | | | |

Higher numbers indicate higher durability.

TABLE 2
The following compositions were prepared by subsequently
dissolving the compounds in water.

| | Inv. ex. 6 | Inv. ex. 7 | Inv. ex. 8 | Inv. ex. 9 | Inv. ex. 10 |
|---|---|---|---|---|---|
| Ingredient | [% by weight] | | | | |
| Citric acid | 2.5 | 5.0 | 5.0 | 5.0 | 5.0 |
| Serine | 5.0 | 5.0 | 2.5 | 10.0 | — |
| Cysteine | — | — | — | — | 2.5 |
| NaOH | pH ad 2.5 | | | | |
| Water | Ad 100.0 | | | | |
| Hair volume | 0.20 | 0.38 | 0.02 | 0.06 | 0.55 |
| after 10 | | | | | |
| washes | | | | | |

Higher numbers indicate higher durability.

### Methods

Indian Frizzy hair (Fischbach und Miller, Laupheim, Germany) was washed with a shampoo available under the tradename Dualsenses Deep Cleansing Shampoo, combed 5-times from each side and allowed to air-dry. The initial R ratio (Rᵢ=width at tip/length) of the hair strand was measured. 10 mL of the compositions above were applied onto the hair streaks, covered, and allowed to process for 20 min. The strand was blow dried. The dry strand was then treated 6-times with a straightening iron at 230° C. and the maximum ratio (Rₘₐₓ) of the hair strand was measured after the treatment.

### Durability Test:

The test strand was washed with the shampoo from above by wetting the hair with warm water for 10 seconds, applying the shampoo for 30 seconds by hand, rinsing the hair with warm water for 30 seconds, combing each side 5-times and then rinse again for 30 seconds. The dripping wet strand was allowed to air dry in a controlled environment at 22° C. and 60% relative humidity. This process was repeated until 10 washes were achieved and the R₁₀ ratio was determined.

### Calculation of Durability:

$$Durability = \frac{{R}_{10} - {R}_{i}}{{R}_{max} - {R}_{i}}$$

with the following definitions:
Each R is the ratio of width at tip to length of a hair streak.
Rᵢ is the initial ratio prior to treatment
Rₘₐₓ is the maximum ratio upon straightening treatment
R₁₀ is the ratio upon 10 washes of the straightened hair streak

1. An aqueous reshaping composition for keratin fibers, having a pH in the range of 1 to 4, and comprising:
a) one or more bidentate or tridentate carboxylic acid, and/or their salt(s), and/or their hydrates, and/or their mixtures; and
b) one or more amino acid(s), and/or their salt(s), and/or their mixtures,
wherein the total concentration of compound(s) according to group a) is 1% by weight or more, calculated to a total weight of the composition.

2. The composition according to claim 1 wherein the one or more compound(s) according to group a) is/are a bidentate carboxylic acid(s) selected from oxalic acid, malonic acid, succinic acid, glutaric acid, adipic acid, pimelic acid, suberic acid, azelaic acid, sebacic acid, maleic acid, fumaric acid, acetylenedicarboxylic acid, glutaconic acid, traumatic acid, tartronic acid, mesoxalic acid, malic acid, tartraric acid, oxaliacetic acid, dioxosuccinic acid, glutaric acid, ketoglutaric acid, and/or their salt(s), and/or their hydrates, and/or their mixtures.

3. The composition according to claim 1, wherein the one or more compound(s) according to group a) is/are a tridentate carboxylic acid selected from citric acid, isocitric acid, aconitic acid, propane-1,2,3-tricarboxylic acid, trimesic acid, and/or their salt(s), and/or their hydrates, and/or their mixtures.

4. The composition according to claim 1, wherein the total concentration of compound(s) according to group a) is in the range of 2% to 30% by weight, calculated to the total weight of the composition.

5. The composition according to claim 1, wherein the one or more compound(s) according to group b) is/are serine, threonine, cysteine, cystine, alanine, glycine, proline, and/or valine, and/or their salt(s), and/or their mixtures.

6. The composition according to claim 1, wherein the weight ratio of compound(s) according to group a) to compound(s) according to group b) is in the range of 0.2 to 5.

7. The composition according to claim 1, wherein the total concentration of the one or more compound(s) according to group b) is in the range of 0.1% to 15% by weight, calculated to the total weight of the composition.

8. The composition according to claim 1, further comprising one or more conditioning compound(s) as compound(s) according to group c), selected from silicones, fatty alcohols, and/or cationic surfactants, and/or their salt(s), and/or their mixtures.

9. The composition according to claim 1, wherein the composition is a hair straightening composition.

10. The composition according to claim 1, wherein the pH of the composition is in the range of 2 to 4.

11. (canceled)

12. A two-chamber packaging device having:
a first chamber comprising a first aqueous composition comprising one or more compound(s) according to group a) as defined in claim 1, and
a second chamber comprising a second aqueous composition comprising one or more compound(s) according to group b) as defined in claim 1.

13. A kit-of-parts having a first component with an aqueous composition comprising one or more compound(s) according to group a) as defined in claim 1, and a second component with an aqueous composition comprising one or more compound(s) according to group b) as defined in claim 1.

14. A reshaping process for keratin fibers comprising the steps in the following order of:
i) providing the composition as defined in claim 1, or providing and mixing the first and second component as defined in claim 13, or providing and dispensing and optionally mixing the compositions as defined in claim 12, and yielding a composition having a pH in the range of 1 to 4,
ii) applying the composition of step i) onto keratin fibers,
iii) optionally covering the keratin fibers with a moisture barrier,
iv) leaving the composition onto the keratin fibers for a time period of 1 min to 24 h,
v) optionally rinsing-off the hair,
vi) drying the hair,
vii) applying heat to the keratin fibers at a temperature in the range of 50° C. to 230° C., and
viii) rinsing-off the keratin fibers and optionally drying the keratin fibers.

15. (canceled)

16. The composition according to claim 1, wherein the one or more compound(s) according to group a) is/are selected from succinic acid, and/or its salt(s), and/or its hydrate(s).

17. The composition according to claim 1, wherein the one or more compound(s) according to group a) is/are citric acid or isocitric acid, and/or their salt(s), and/or their hydrates, and/or their mixtures.

18. The composition according to claim 1, wherein the total concentration of compound(s) according to group a) is in the range of 3% to 8% by weight, calculated to the total weight of the composition.

19. The composition according to claim 1, wherein the one or more compound(s) according to group b) is/are serine and/or threonine, and/or their mixtures, and/or their salt(s).

20. The composition according to claim 1, wherein the weight ratio of compound(s) according to group a) to compound(s) according to b) is in the range of of 0.5 to 1.

21. The composition according to claim 1, wherein the total concentration of the one or more compound(s) according to group b) is in the range of 2% to 8% by weight, calculated to the total weight of the composition.
